(19) United States
(12) Patent Application Publication
(10) Pub. No.: US 2022/0030844 A1
(43) Pub. Date: Feb. 3, 2022

(54) VOLATILE SUBSTANCE EVAPORATION DEVICE

(71) Applicant: ZOBELE HOLDING SPA, TRENTO (IT)

(72) Inventors: Montserrat Riera Giner, Barcelona (ES); Moisés Caballero Tapia, Barcelona (ES); Alba Graus Ferrer, Barcelona (ES)

(21) Appl. No.: 17/276,977

(22) PCT Filed: Sep. 17, 2019
(86) PCT No.: PCT/EP2019/074819

§ 371 Date: Mar. 17, 2021

(30) Foreign Application Priority Data
Sep. 17, 2018 (ES) P201830894

(51) Int. Cl. A01M 1/20

(52) U.S. Cl. CPC A01M 1/2055

(57) ABSTRACT

The volatile substance evaporation device comprises a casing (1) provided with an evaporation surface (2), said casing (1) housing inside it a substrate (3) impregnated with said volatile substances, wherein said substrate (3) is a sheet which preferably comprises a plurality of grooves. It allows the device to have a similar diffusion value for a specific air flow and for a considerable internal temperature of the vehicle, thus ensuring continuous evaporation.

The present invention relates to a volatile substance evaporation device, in particular for its use in vehicles.

### BACKGROUND OF THE INVENTION

At present, there are various volatile substance evaporation devices for use in the interior of vehicles.

Many of these devices are characterised by functioning statically, with a large evaporation surface, or by being positioned in a ventilation outlet and using the air to evaporate the volatile substances.

Normally, the vast majority of products for vehicles are products with high and medium volatility, and those which have low volatility (complex perfumes or insecticides) are characterised by being connected to a power source to generate sufficient heat required for their evaporation and control.

Volatile substance evaporation devices which are placed in ventilation outlets are characterised by being very well designed to control the evaporation with the air flow, but difficult to control passively when there is no flow.

In addition, these evaporation devices are also characterised by being very variable depending on the temperature of the vehicle.

Therefore, an object of the present invention is to provide a device for evaporating volatile substances, mainly those with low volatility, which does not require a power source and which has a similar diffusion value for a specific air flow and for a considerable internal temperature of the vehicle, thus ensuring continuous evaporation both if the air flow generated by the ventilation outlets of the vehicle is functioning or if the vehicle is exposed to the sun.

### DESCRIPTION OF THE INVENTION

The evaporation device of the invention of the invention resolves the aforementioned drawbacks and has other advantages which are described below.

The volatile substance evaporation device according to the present invention comprises a casing provided with an evaporation surface which houses inside it a substrate impregnated with said volatile substances, wherein said substrate is a sheet which is preferably provided with a plurality of grooves.

When the vehicle is at high temperatures (for example 45° C.), this substrate allows for a value to be obtained that is very similar to when an air flow is made to pass through (for example 1 m/s).

In this second case, the volatile substance release value is determined by the thickness or grammage of the substrate and by the grooves that the substrate has, which allow the air flow to pass therethrough, releasing the volatile substances.

Therefore, whether the vehicle is exposed to the sun or the air flow of the ventilation of the vehicle is in operation, we obtain similar diffusion values.

According to one preferred embodiment of the device according to the present invention, said substrate is a non-woven fabric. However, it should be indicated that said substrate could be made of a different material, for example ceramic.

Advantageously, said grooves are placed parallel to one another, for example forming two rows of grooves. It should be indicated that said grooves are grooves or through-holes, i.e. they fully pass through said substrate.

Preferably, said evaporation surface is formed by a grid.

According to a preferred embodiment, the grammage of said non-woven fabric is between 30 and 40 g/m² and said grooves are placed parallel to one another at a distance comprised between 2.5 and 3 mm.

In order to ensure the correct fixing of the substrate on the casing, said substrate comprises a plurality of fixing holes which match fixing holes in the casing.

The volatile substance evaporation device according to the present invention can also comprise a fastening clip on its casing for coupling or fastening thereof to a grid or ventilation outlet of a vehicle.

### BRIEF DESCRIPTION OF THE DRAWINGS

In order to better understand what has been set forth above, several drawings schematically depicting only by way of non-limiting example a practical embodiment are attached.

FIG. 1 is an exploded perspective view of the volatile substance evaporation device according to the present invention;

FIG. 2 is a plan view of the substrate impregnated in the volatile substances of the evaporation device according to the present invention; and

FIG. 3 is a perspective view of the volatile substance evaporation device according to the present invention.

### DESCRIPTION OF A PREFERRED EMBODIMENT

As is shown in the figures, the volatile substance evaporation device according to the present invention comprises a casing 1 provided with an evaporation surface 2, such as for example a grid, and houses in its interior a substrate 3 impregnated in said volatile substances.

Said casing 1 also preferably comprises a fastening clip 4 to fix the device to a ventilation outlet of a vehicle.

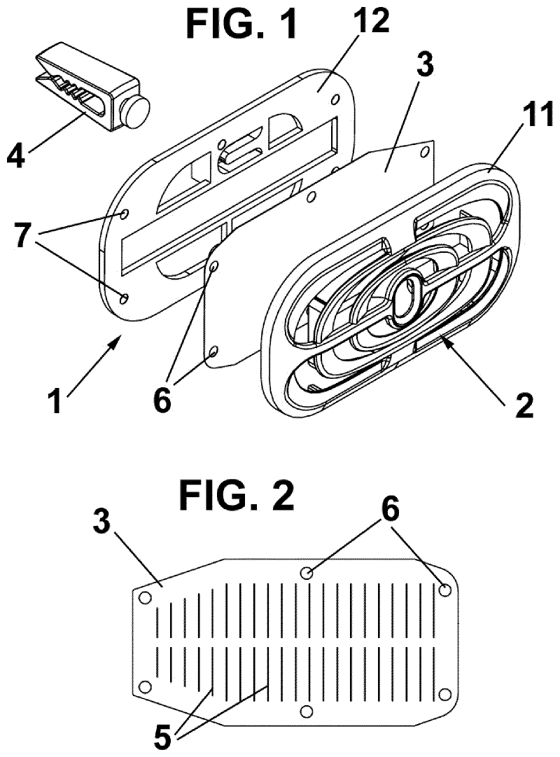

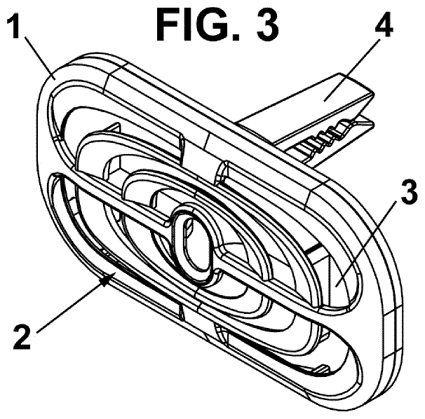

According to the embodiment shown in FIG. 1, said casing 1 is formed by a front half 11 and a back half 12 which, in their position of use (FIG. 3), are joined together.

The substrate 3 is in the form a sheet, that is to say, thin and occupying a substantial surface, and is preferably formed of non-woven fabric, of any type known by the person skilled in the art, although it could be made of another material, for example ceramic.

Said substrate 3 comprises a plurality of grooves 5, i.e. grooves or through-holes which pass through said substrate 3. It should be indicated that, for the sake of simplicity, the grooves 5 of the substrate 3 have only been represented in FIG. 2.

According to a preferred embodiment shown in FIG. 2, said grooves 5 are arranged parallel to one another and defining two rows of grooves 5, although they could be placed in another suitable manner.

In order to ensure the correct fixing of the substrate 3 on the casing 1, said substrate 3 comprises a plurality of fixing holes 6 which match fixing holes 7 in the casing 1, for example in the back half 12 of the casing 1, as shown in FIG. 1. In this way, the substrate 3 is fixed in position by means of complementary protrusions (not shown in the figures) of the front half 11 of the casing 1.

In addition, this fixing system and the grid configuration of the evaporation surface 2 prevents the substrate 3 from rippling due to its thinness when the air flow passes.

Only as an example, a non-woven substrate 1 with a surface of 2045 mm² and a grammage of approximately 35 g/cm² can be used, with two parallel rows of grooves 5 with a distance of 2.8 mm between each groove.

Evaporations of a formulation (for example insecticide and solvents) have been made with a low-volatility component and the following loss values in weight of the substrate 3 are observed, which are very similar under a flow of 1 m/s or statically at a temperature of 45° C.

It should be indicated that the evaporation of insecticide is maintained in a similar manner in both cases and in the desired range, generating the efficiency required of the product, with a target duration of 320 hours.

Despite the fact that reference has been made to a specific embodiment of the invention, it is evident for the person skilled in the art that numerous variations and changes may be made to the volatile substance evaporation device described, and that all the aforementioned details may be substituted by other technically equivalent ones, without detracting from the scope of protection defined by the attached claims.

1. A volatile substance evaporation device, comprising:
a casing provided with an evaporation surface, said casing housing inside it a substrate impregnated with volatile substances, wherein said substrate is a sheet.

2. The volatile substance evaporation device according to claim 1, wherein said substrate comprises a plurality of grooves formed therein.

3. The volatile substance evaporation device according to claim 1, wherein said substrate is made of non-woven fabric.

4. The volatile substance evaporation device according to claim 2, wherein said grooves are placed parallel to one another.

5. The volatile substance evaporation device according to claim 2, wherein said grooves include two rows of grooves.

6. The volatile substance evaporation device according to claim 1, wherein said evaporation surface is formed by a grid.

7. The volatile substance evaporation device according to claim 3, wherein a grammage of said non-woven fabric is between 30 and 40 g/m².

8. The volatile substance evaporation device according to claim 4, wherein said grooves are placed parallel to one another at a distance between 2.5 and 3 mm.

9. The volatile substance evaporation device according to claim 1, wherein said substrate comprises a first plurality of fixing holes which match a second plurality of fixing holes formed in the casing (1).

10. The volatile substance evaporation device according to claim 1, wherein said casing comprises a fastening clip.

11. The volatile substance evaporation device according to claim 2, wherein said substrate is made of non-woven fabric.

12. The volatile substance evaporation device according to claim 11, wherein a grammage of said non-woven fabric is between 30 and 40 g/m².

13. The volatile substance evaporation device according to claim 4, wherein said grooves include two rows of grooves.
